(19) United States
(12) Patent Application Publication
(10) Pub. No.: US 2018/0289729 A1
(43) Pub. Date: Oct. 11, 2018

(54) COMBINATION THERAPY FOR TREATMENT OF HUMAN NEURODEGENERATIVE DISEASES

(71) Applicant: Robert John Petcavich, The Woodlands, TX (US)

(72) Inventor: Robert John Petcavich, The Woodlands, TX (US)

(21) Appl. No.: 15/947,370

(22) Filed: Apr. 6, 2018

Related U.S. Application Data
(60) Provisional application No. 62/483,069, filed on Apr. 7, 2017.

(51) Int. Cl. A61K 31/706; A61P 25/28; A61K 9/00

(52) U.S. Cl. CPC A61K 31/706; A61P 25/28; A61K 31/22; A61K 2300/00; A61K 9/0053

(57) ABSTRACT

The present disclosure provides a combination therapy to treat neurodegenerative diseases. In one embodiment, Alzheimer's disease is treated with an orally administered mitochondria stimulant, neuro protective antioxidant that crosses the blood brain barrier and is supplemented with a drug that promotes hippocampal neurogenesis. The combination therapy of a mitochondria stimulant, a brain available antioxidant and a neurogenesis agonist, may be employed to prevent, inhibit or treat neurodegenerative diseases that have a root cause of inflammation, oxidative stress, followed by free radical biologic macromolecule destruction, and eventually cell death, such as Parkinson's, age related Dementia, Multiple Sclerosis (MS) and Amyotrophic Lateral Sclerosis (ALS).

### CROSS-REFERENCE TO RELATED APPLICATIONS

This application claims the benefit of the filing date of U.S. application Ser. No. 62/483,069, filed on Apr. 7, 2017, the disclosure of which is incorporated by reference herein.

### FIELD OF THE INVENTION

The present disclosure provides a therapeutic combination approach for the treatment of chronic neurodegenerative diseases. In one embodiment, one or more mitochondria stimulants, and one or more neuroprotective antioxidants are used in combination with one or more neurogenesis agonists that replace or rebuild the damaged neuron population of interest, to prevent, inhibit or treat neurodegenerative diseases such as Alzheimer's disease.

### BACKGROUND

Neurodegeneration is the progressive loss of structure or function of neurons, including death of neurons. Many neurodegenerative diseases including amyotrophic lateral sclerosis (ALS), Parkinson's, Alzheimer's (AD), and Huntington's occur as a result of neurodegenerative processes. Such diseases are incurable, resulting in progressive degeneration and/or death of neuron cells. As research progresses, many similarities appear that relate these diseases to one another on a sub-cellular level. Discovering these similarities offers hope for therapeutic advances that could ameliorate many diseases simultaneously. There are many parallels between different neurodegenerative disorders including atypical protein assemblies as well as induced cell death. Neurodegeneration can be found in many different levels of neuronal circuitry ranging from molecular to systemic.

AD is the most common cause of dementia in people over 65 years of age; including more than 20 million people are affected by sporadic AD worldwide. As the life span of the world's population increases due to better lifestyle choices and medical advances AD will become a very serious public health issue with enormous social and economic costs. AD is characterized by aggregated protein that forms amyloid plaques and neurofibrillary tangles (NTFs), causing brain atrophy and cognitive impairment progression leading to dementia. Protein's aggregates are a common feature of several neurodegenerative diseases, however, some cellular accumulated proteins in the senescent brains, do not cause cognitive disorders, suggesting that, this might be the result of normal aging processes. AD has an important vascular component and recently it has been considered that early detection of proto-fibrillar amyloid in young Familiar AD (FAD) patients by Positron Emission Tomography (PET) could be an important biomarker for the prevention of vascular deterioration (VaD), as the common cause of AD. Nevertheless, aggregation of the tau protein is an exception. Studies that evaluate the brains of AD patients have found that the number of NTFs correlates with dementia more accurately than the amyloid plaques in the later stage. This evidence generates great interest for therapeutic intervention in AD and other tauopathies caused by chronic or acute injuries such as stroke.

Alzheimer's disease has been hypothesized to be a protein misfolding disease (proteopathy), caused by accumulation of abnormally folded A-beta and tau proteins in the brain. Plaques are made up of small peptides, 39-43 amino acids in length, called beta-amyloid (also written as A-beta or Aβ). Beta-amyloid is a fragment from a larger protein called amyloid precursor protein (APP), a transmembrane protein that penetrates through the neuron's membrane. APP is critical to neuron growth, survival and post-injury repair. In Alzheimer's disease, an unknown process causes APP to be divided into smaller fragments by enzymes through proteolysis. One of these fragments gives rise to fibrils of beta-amyloid, which form clumps that deposit outside neurons in dense formations known as senile plaques.

The greatest risk factor for neurodegenerative diseases is aging. Mitochondrial DNA mutations as well as oxidative stress both contribute to aging. Many of these diseases are late-onset, meaning there is some factor that changes as a person ages for each disease. One constant factor is that in each disease, neurons gradually lose function as the disease progresses with age. Once AD is initiated in the brain it is the hypothesis of the present inventor that the process is autocatalytic meaning that inflammation induced free radicals degrade and cross link critical proteins such as APP which are then unable to be cleared by proteolytic enzymes, the NTFs propagate and accumulate intracellular and extracellularly eventually resulting in local and near proximity cell death. The region of the brain most affected is the Hippocampus where most memory functions arise and are stored in humans. Once the process has begun it can then spread and cause loss of neurons and synapses in the cerebral cortex and certain subcortical regions. This loss results in gross atrophy of the affected regions, including degeneration in the temporal lobe and parietal lobe, and parts of the frontal cortex and cingulate gyrus.

### SUMMARY

The present disclosure provides for, in one embodiment, the inhibition or the treatment of neurodegenerative diseases such as AD with a combination treatment that enhances mitochondria function, arrests free radical protein degradation and cross-linking and/or stimulates neurogenesis, e.g., of the affected brain region, with an agonist. The combination treatment allows for prevention, inhibition or treatment of free radical oxidation and oxidative stress caused by chronic brain inflammation resulting in neuron death.

In one embodiment, a combination therapy for the treatment of neurodegenerative diseases is provided that utilizes one or more mitochondria energy enhancers, one or more antioxidants that cross the blood brain barrier and/or one or more neurogenesis agonists.

In one embodiment, a combination therapy for the treatment of Alzheimer's disease is provided that utilizes one or more mitochondria energy enhancers, one or more antioxidants that cross the blood brain barrier and/or one or more hippocampus neurogenesis agonists that cross the blood brain barrier. Stimulation of hippocampal neurogenesis replaces damaged or dead neurons.

In one embodiment, a method of treating a neurodegenerative disease is provided. In one embodiment, the method includes administering to a subject having a neurodegenerative disease, e.g., a mammal such as a human having a neurodegenerative disease, an effective amount of a combination of at least two of: i) one or more mitochondria stimulants, ii) one or more antioxidants that cross the blood brain barrier, or iii) one or more neurogenesis agonists. The two or more of mitochondria stimulant, antioxidant, or neurogenesis agonist may be administered sequentially (in any order) or contemporaneously. In one embodiment, a composition comprising one or more mitochondria stimulants and one or more antioxidants that cross the blood brain barrier is administered. In one embodiment, a composition comprising one or more mitochondria stimulants, and one or more neurogenesis agonists is administered. In one embodiment, a composition comprising one or more antioxidants that cross the blood brain barrier and one or more neurogenesis agonists is administered. In one embodiment, a composition comprising one or more mitochondria stimulants, one or more antioxidants that cross the blood brain barrier, and one or more neurogenesis agonists is administered. In one embodiment, a composition comprising one or more mitochondria stimulants and a composition comprising one or more antioxidants that cross the blood brain barrier are administered. In one embodiment, a composition comprising one or more mitochondria stimulants and a composition comprising one or more neurogenesis agonists are administered. In one embodiment, a composition comprising one or more antioxidants that cross the blood brain barrier and a composition comprising one or more neurogenesis agonists are administered. In one embodiment, a composition comprising one or more mitochondria stimulants, a composition comprising one or more antioxidants that cross the blood brain barrier, and a composition comprising one or more neurogenesis agonists are administered. In one embodiment, the neurodegenerative disease is Alzheimer's disease. In one embodiment, the mitochondria stimulant comprises nicotinamide mononucleotide (NMN) or an analog thereof, e.g., thio-NMN, 3-AcPyr-MN or 3-OHCPyr-MN. In one embodiment, the mitochondria stimulant is administered in an amount from about 50 milligrams to about 2500 milligrams per day. In one embodiment, the mitochondria stimulant is administered in an amount from about 100 milligrams to about 1000 milligrams per day or about 200 milligrams to about 800 milligrams per day. In one embodiment, the antioxidant comprises N-acetyl ethyl ester, a cell permeable analog of glutathione, e.g., glutathione ethyl ester, hydroxytyrosol or genestein, or an analog thereof, e.g., 7-difluoromethoxyl-5,4′-di-n-octylgenistein or daidzein. In one embodiment, the antioxidant is administered in an amount from about 25 milligrams to about 7500 milligrams per day. In one embodiment, the antioxidant is administered in an amount from about 100 milligrams to about 1500 milligrams per day or about 200 milligrams to about 1200 milligrams per day. In one embodiment, the neurogenesis agonist is a kinase Alk5 inhibitor. In one embodiment, the neurogenesis agonist comprises 2-(3-(6-methylpyridin-2-yl)-1H-pyrazol-4-yl)-1,5-naphthyridine, [(6aR)-trans-3-(1,1-dimethylheptyl)-6a, 7,10,10a-tetrahydro-1-hydroxy-6, 6-dimethyl-6H-dibenzo [b,d] pyran-9-methanol], GSK 1838705, ALX-270-448, SB431542, KRCA 0008, ASP 3026, PF 0646392, criztinib, EW-7197, or anatamide. In one embodiment, the neurogenesis agonist is administered in an amount from about 0.0005 μM to about 20 μM per day. In one embodiment, the neurogenesis agonist is administered in an amount from about 0.001 μM to about 10 μM per day or about 0.05 μM to about 5 μM. In one embodiment, the combination is orally administered. In one embodiment, at least one of the combination is orally administered. In one embodiment, at least one of the combination is in sustained release dosage form.

In one embodiment, a method of treating neurodegenerative disease is provided that includes administering to a subject having a neurodegenerative disease an effective amount of a combination of at least two of: i) one or more mitochondria stimulants, ii) rapamycin, or iii) one or more antioxidants that cross the blood brain barrier. The two or more of mitochondria stimulant, antioxidant, or rapamycin may be administered sequentially (in any order) or contemporaneously. In one embodiment, the neurodegenerative disease is Alzheimer's disease. In one embodiment, the mitochondria stimulant comprises nicotinamide mononucleotide. In one embodiment, the mitochondria stimulant is administered in an amount from about 50 to about 2500 milligrams per day. In one embodiment, the mitochondria stimulant is administered in an amount from about 100 milligrams to about 1000 milligrams per day or about 200 milligrams to about 800 milligrams per day. In one embodiment, the antioxidant comprises N-acetyl ethyl ester, glutathione ethyl ester, hydroxytyrosol or genestein. In one embodiment, the antioxidant is administered in an amount from about 25 to about 7500 milligrams per day. In one embodiment, the antioxidant is administered in an amount from or about 50 to about 5000 milligrams per day, from about 100 milligrams to about 1500 milligrams per day or about 200 milligrams to about 1200 milligrams per day. In one embodiment, the combination is orally administered. In one embodiment, at least one of the combination is orally administered. In one embodiment, at least one of the combination is in sustained release dosage form. In one embodiment, the method further comprises administering one or more neurogenesis agonists.

Also provided is a pharmaceutical composition comprising at least two of: i) one or more mitochondria stimulants, ii) one or more antioxidants that cross the blood brain barrier, or iii) one or more neurogenesis agonists. In one embodiment, the pharmaceutical composition has about 50 milligrams to about 2500 milligrams of the mitochondria stimulant in one embodiment, the pharmaceutical composition has about 25 milligrams to about 7500 milligrams of the antioxidant.

Further provided is a pharmaceutical composition comprising at least two of: i) one or more mitochondria stimulants, ii) rapamycin, or iii) one or more antioxidants that cross the blood brain barrier. In one embodiment, the pharmaceutical composition has about 50 milligrams to about 2500 milligrams of the mitochondria stimulant. In one embodiment, the pharmaceutical composition has about 25 milligrams to about 7500 milligrams of the antioxidant.

### DETAILED DESCRIPTION

The following discussion is directed towards the various embodiments of the invention. Although one or more of these embodiments may be preferred, the embodiments disclosed should not be interpreted, or otherwise used, as a limiting the scope of the disclosure, including the claims. In addition one skilled in the art will understand that the following description has broad application, and the discussion of any embodiment is meant only to be exemplary of that embodiment, and not intended to intimate that the scope of the disclosure, including the claims, is limited to that embodiment.

Mitochondrial dysfunction is a hallmark of neurodegenerative diseases with morphological and functional abnormalities limiting the electron transport chain and adenosine triphosphate (ATP) production as seen in AD. Nicotinamide adenine dinucleotide (NAD) is a cofactor that is essential for many biological reactions in either oxidized (NAD+) or reduced (NADH) forms. NAD+ and NADH mediate reductive metabolic reactions. NAD+ is essential to many mitochondrial enzymatic reactions and appropriate bioenergetics metabolism. NAD levels naturally decline with aging. In normal conditions the loss of NAD+ inhibits cellular respiration, resulting in loss of ATP production and eventually cell death followed by oxidative stress and inflammation to clear cell debris. Preventing and or supplementing NAD+ depletion and cellular energy deficits is part of the current strategy of the present invention to arrest and modulate inflammation and oxidative stress as the first step in neuroprotective therapy for neurodegenerative diseases. Therefore supplementing with nicotinamide mononucleotide from the range of about 500 to about 2500 milligrams per day or about 100 to about 2000 milligrams per day, e.g., about 250 to about 750 milligrams, is part of the treatment. It has been shown in prior art that Alzheimer's disease transgenic relevant mice (Long et al., 2015) responded positively to NAD supplements and down regulation of AD associated generation of protein plaques.

Another hallmark of neurodegenerative diseases is that they all appear to have a root cause of chronic inflammation possibly aggravated by astrocyte and/or microglial cells. In the case of AD there also appears to be a link to brain cell glucose uptake and metabolism or lack thereof and therefore a suspected link to diabetes. Without sufficient glucose uptake cells will eventually die and need to be cleared by the brains immune system. Cell death results the release of signaling molecules that can begin an autocatalytic cascade of neighboring cell apoptosis. During this process reactive oxygen species are generated that results in free radical destruction or crosslinking of cellular macromolecules. The destruction results in inflammation and crosslinking of proteins which gives rise to the accumulation of plaques or fibrous entanglements which are present in neurodegenerative diseases such as AD and Parkinson's disease

Therefore, in one embodiment, the generation of destructive free radicals is inhibited by flooding the brain with free radical inhibitors. However, because the blood brain barrier (BBB) prevents many molecules from passing from the blood into the brain there are not many orally delivered options available to be transported across the BBB to modulate free radical generation and destruction due to chronic inflammation. It is desirable to have an antioxidant that can be ingested orally versus by intravenous or via spinal fluid delivery, which makes it painful and inconvenient. U.S. Pat. No. 5,874,468, which is incorporated by reference herein, teaches brain target hydrophobic antioxidants for the treatment of neurodegenerative diseases. However, the only testing was conducted in vitro on PC12 rat brain cells (Offen et at, 1996) and in vivo in mice to demonstrate that the molecules of interest cross the BBB of those mammals. Historically it has been found that lab results from rat and mice models rarely translate to successful human therapies. Subsequently (Bahet-Stroomza et al., 2005) demonstrated that one of the compounds in U.S. Pat. No. 5,874,468 was effective in preventing oxidative damage in an in vivo Parkinson's rat model. The molecule did penetrate the rat BBB and did have a therapeutic effect. However there was no teaching of any efficacy on human in vivo or in vitro neuron models.

The antioxidant molecules disclosed in U.S. Pat. No. 5,847,468 may be effective as human therapeutics taken in doses from about 25 to about 7500 milligrams per day or about 50 to about 5000 milligrams per day, e.g., about 250 to about 1000 milligrams per day. In one embodiment, the antioxidant includes N-acetyl ethyl ester, glutathione ethyl ester, hydroxytyrosol from the o-diphenol family, and genestein. In one embodiment, time-release capsules that may or may not have enteric release properties are used to deliver all the antioxidants orally. Another option is to administer rapamycin to suppress the immune system and therefore oxidative stress caused by immune cells, with or without the use of antioxidants. Rapamycin may be administered from about 0.001 to about 10 micro molar (μM) concentrations.

Other molecules employed in the method promote neurogenesis of the brain region that is being affected by inflammation and cell death. Some studies suggest that decreased hippocampal neurogenesis can lead to development of Alzheimer's disease (AD). Yet, others hypothesize that AD patients have increased neurogenesis in the CA1 region of Ammon's horn (the principal region of AD hippocampal pathology) in order to compensate for neuronal loss. While the exact nature of the relationship between neurogenesis and Alzheimer's disease is unknown, insulin-like growth factor 1-stimulated neurogenesis produces major changes in hippocampal plasticity and seems to be involved in Alzheimer's pathology. Yet additional signals indicate that diabetes and insulin management may play a role in the onset and progression of AD. Eph receptors and ephrin signaling have been shown to regulate adult neurogenesis in the hippocampus and have been studied as potential targets to treat some symptoms of AD.

Neurogenesis is the process of birth of neurons wherein neurons are generated from neural stem cells. Contrary to popular belief, neurogenesis continuously occurs in specific regions in the adult brain. Developmental neurogenesis and adult neurogenesis differ markedly. This discussion is limited in scope to adult neurogenesis. In humans, new neurons are continually born throughout adulthood in two regions of the brain: The sub granular zone (SGZ), part of the dentate gyrus of the hippocampus. The striatum; however the adult-born neurons are a type of interneuron, not a type that projects to other brain areas. Adult neurogenesis poses many implications in terms of its functioning in learning and memory, emotion, stress, depression, and other conditions.

Therefore, in addition to enhancing mitochondria function, and arresting or preventing oxidative stress, hippocampal neurogenesis stimulated to replace dead or dying cells. A number of potential agonists have been reported, see, e.g., Yousef et al., 2015), including Kinase Alk5 inhibitor 2-(3-(6-methylpyricin-2-yl)-1H-pyrazol-4-yl)-1,5-naphthyridine. That particular agonist was demonstrated to enhance hippocampal neurogenesis in both young and “old” mice models. It was observed that there was a two-fold increase in the number of proliferating neural stem and progenitor cells within the hippocampal dentate gyrus. The daily dose can range from about 0.0005 to about 20 μM or about 0.001 to about 10 μM, e.g., about 0.1 to about 1 μM. Additionally it has been shown that certain cannabinoids can induce hippocampal neurogenesis. (Jiang et al, 2015) teaches the role of cannabinoid receptors and various cannabinoids in particular the dramatic effect that [(6aR)-trans-3-(1,1-dimethylheptyl)-6a, 7,10,10a-tetrahydro-1-hydroxy-6, 6-dimethyl-6H-dibenzo [b,d] pyran-9-methanol] has on hippocampal neurogenesis in rat models. The aforementioned hippocampal neurogenesis agonists are just two examples of therapeutics that can be utilized in the present invention to stimulate hippocampal neurogenesis.

There appears to be a lot of direct and indirect evidence of glucose uptake or lack thereof and its metabolism by brain cells and a possible link in AD. Therefore diabetes and insulin regulation may play a critical role in modulating the disease progression therefore an optional additional step to the present invention would be to treat the disease with well known diabetes therapeutics such as Metformin, Extendin-4, Victoza, Lyxuma, Tarzeum, Trulicity, all well known FDA approved therapeutics used to treat Diabetes but not used to treat neurodegenerative diseases in particular AD.

Administration of compositions having one or more mitochondria stimulants, one or more antioxidants that cross the blood brain barrier, one or more neurogenesis agonists, rapamycin, or a diabetic therapeutic, or any combination thereof, can be via any of suitable route of administration, particularly parenterally, for example, orally, intravenously, intra-arterially, intraperitoneally, intrathecally, intraventricularly, intraurethrally, intrasternally, intracranially, intramuscularly, or subcutaneously. Such administration may be as a single dose or multiple doses, or as a short- or long-duration infusion. Implantable devices (e.g., implantable infusion pumps) may also be employed for the periodic parenteral delivery over time of equivalent or varying dosages of the particular formulation. For such parenteral administration, the compounds may be formulated as a sterile solution in water or another suitable solvent or mixture of solvents. The solution may contain other substances such as salts, sugars (particularly glucose or mannitol), to make the solution isotonic with blood, buffering agents such as acetic, critric, and/or phosphoric acids and their sodium salts, and preservatives.

The compositions invention alone or in combination with other active agents can be formulated as pharmaceutical compositions and administered to a vertebrate host, such as a human patient in a variety of forms adapted to the chosen route of administration, e.g., orally or parenterally, by intravenous, intramuscular, topical or subcutaneous routes.

Thus, the compositions may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the vertebrate's diet. For oral therapeutic administration, the composition optionally in combination with another active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of the compound and optionally other active compound in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, one or more mitochondria stimulants, one or more antioxidants that cross the blood brain barrier, one or more neurogenesis agonists, rapamycin, or a diabetic therapeutic optionally in combination with another active compound may be incorporated into sustained-release preparations and devices.

The composition optionally in combination with another active compound may also be administered intravenously or intraperitoneally by infusion or injection. Solutions of one or more mitochondria stimulants, one or more antioxidants that cross the blood brain barrier, one or more neurogenesis agonists, rapamycin, or a diabetic therpeutic, or any combination thereof, optionally in combination with another active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms during storage can be brought about by various antibacterial and antifungal agents, for example, parabens, chlorobutanol, phenol, sorbic acid, thimerosal, and the like. In many cases, it may be useful to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating compounds in the required amount in the appropriate solvent with various of the other ingredients enumerated above, as required, followed by filter sterilization. In the case of sterile powders for the preparation of sterile injectable solutions, one method of preparation includes vacuum drying and the freeze drying techniques, which yield a powder of the active ingredient plus any additional desired ingredient present in the previously sterile-filtered solutions.

For topical administration, the compounds optionally in combination with another active compound may be applied in pure form, e.g., when they are liquids.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

In addition, in one embodiment, the pharmaceutical composition provides various dosage formulations of the compound(s) optionally in combination with another active compound for inhalation delivery. For example, formulations may be designed for aerosol use in devices such as metered-dose inhalers, dry powder inhalers and nebulizers.

Useful dosages can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compounds optionally in combination with another active compound in a liquid composition, may be from about 0.1-25 wt-%, e.g., from about 0.5-10 wt-%. The concentration in a semi-solid or solid composition such as a gel or a powder may be be about 0.1-5 wt-%, e.g., about 0.5-2.5 wt-%.

The active ingredient may be administered to achieve peak plasma concentrations of the active compound of, in one embodiment, from about 0.5 to about 75 μM, e.g., about 1 to 50 μM, such as about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The amount of the compounds optionally in combination with another active compound, or an active salt or derivative thereof, required for use in treatment may vary not only with the particular salt selected but also with the route of administration, the nature of the condition being treated and the age and condition of the patient and will be ultimately at the discretion of the attendant physician or clinician. In general, however, a suitable dose may be in the range of from about 0.5 to about 100 mg/kg, e.g., from about 10 to about 75 mg/kg of body weight per day, such as 3 to about 50 mg per kilogram body weight of the recipient per day, for instance in the range of 6 to 90 mg/kg/day, e.g., in the range of 15 to 60 mg/kg/day.

The compound(s) optionally in combination with another active compound may be conveniently administered in unit dosage form; for example, containing 5 to 1000 mg, conveniently 10 to 750 mg, most conveniently, 50 to 500 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye. The dose, and perhaps the dose frequency, will also vary according to the age, body weight, condition, and response of the individual vertebrate. In general, the total daily dose range for an active agent for the conditions described herein, may be from about 50 mg to about 5000 mg, in single or divided doses. In one embodiment, a daily dose range should be about 100 mg to about 4000 mg, e.g., about 1000-3000 mg, in single or divided doses, e.g., 750 mg every 6 hr of orally administered compound. This can achieve plasma levels of about 500-750 μM, which can be effective to kill cancer cells. In managing the patient, the therapy should be initiated at a lower dose and increased depending on the patient's global response.

In one embodiment, a method of treating a neurodegenerative disease is provided. The method includes administering to a subject having a neurodegenerative disease an effective amount of a combination of one or more mitochondria stimulants, one or more antioxidants that cross the blood brain barrier, and one or more neurogenesis agonists. In one embodiment, the neurodegenerative disease is Alzheimer's disease. In one embodiment, the mitochondria stimulant comprises nicotinamide mononucleotide. In one embodiment, the mitochondria stimulant is administered in an amount from about 50 milligrams to about 2500 milligrams per day. In one embodiment, the mitochondria stimulant is administered in an amount from about 100 milligrams to about 1000 milligrams per day or about 200 milligrams to about 800 milligrams per day. In one embodiment, the antioxidant comprises N-acetyl ethyl ester, glutathione ethyl ester, hydroxytyrosol or genestein. In one embodiment, the antioxidant is administered in an amount from about 25 milligrams to about 7500 milligrams per day. In one embodiment, The antioxidant is administered in an amount from about 100 milligrams to about 1500 milligrams per day or about 200 milligrams to about 1200 milligrams per day. In one embodiment, the neurogenesis agonist comprises 2-(3-(6-methylpyricin-2-yl)-1H-pyrazol-4-yl)-1,5-naphthyridine, [(6aR)-trans-3-(1,1-dimethylheptyl)-6a, 7,10,10a-tetrahydro-1-hydroxy-6, 6-dimethyl-6H-dibenzo [b,d] pyran-9-methanol] or anatamide. In one embodiment, the neurogenesis agonist is administered in an amount from about 0.0005 μM to about 20 μM per day. In one embodiment, the neurogenesis agonist is administered in an amount from about 0.001 μM to about 10 μM per day or about 0.05 μM to about 5 μM. In one embodiment, the combination is orally administered. In one embodiment, at least one of the combination is orally administered. In one embodiment, at least one of the combination is in sustained release dosage form. In one embodiment, the combination includes nicotinamide mononucleotide, one or more of N-acetyl ethyl ester, glutathione ethyl ester, hydroxytyrosol or genestein and one or more of comprises 2-(3-(6-methylpyricin-2-yl)-1H-pyrazol-4-yl)-1,5-naphthyridine, [(6aR)-trans-3-(1,1-dimethylheptyl)-6a, 7,10,10a-tetrahydro-1-hydroxy-6, 6-dimethyl-6H-dibenzo [b,d] pyran-9-methanol] or anatamide.

Further provided is a method of treating neurodegenerative disease that includes administering to a subject having a neurodegenerative disease an effective amount of a combination of one or more mitochondria stimulants, rapamycin, and one or more antioxidants that cross the blood brain barrier. In one embodiment, the neurodegenerative disease is Alzheimer's disease. In one embodiment, the mitochondria stimulant comprises nicotinamide mononucleotide. In one embodiment, the mitochondria stimulant is administered in an amount from about 50 to about 2500 milligrams per day. In one embodiment, the mitochondria stimulant is administered in an amount from about 100 milligrams to about 1000 milligrams per day or about 200 milligrams to about 800 milligrams per day. In one embodiment, the antioxidant comprises N-acetyl ethyl ester, glutathione ethyl ester, hydroxytyrosol or genestein. In one embodiment, the antioxidant is administered in an amount from about 25 to about 7500 milligrams per day. In one embodiment, the antioxidant is administered in an amount from or about 50 to about 5000 milligrams per day, from about 100 milligrams to about 1500 milligrams per day or about 200 milligrams to about 1200 milligrams per day. In one embodiment, the combination is orally administered. In one embodiment, at least one of the combination is orally administered. In one embodiment, at least one of the combination is in sustained release dosage form. In one embodiment, the combination includes nicotinamide mononucleotide, one or more of N-acetyl ethyl ester, glutathione ethyl ester, hydroxytyrosol or genestein and one or more of comprises 2-(3-(6-methylpyricin-2-yl)-1H-pyrazol-4-yl)-1,5-naphthyridine, [(6aR)-trans-3-(1,1-dimethylheptyl)-6a, 7,10,10a-tetrahydro-1-hydroxy-6, 6-dimethyl-6H-dibenzo [b,d] pyran-9-methanol] or anatamide.

### References

### Aaron N Long, Katrina Owens, Anna E Schlappal, Tibor Kristian, Paul S Fishman and Rosemary A Schuh, BMC Neurology (2015) 15:19

Offen, D., Ziv, I., Sternin, H., Melamed, E., and Hochman, A., Exp. Neurol., 141, 32-39.
Hanadie Yousef, Michael J. Conboy, Adam Morgenthaler, Christina Schlesinger, Lukasz Bugaj, Preeti Paliwal, Christopher Greer, Irina M. Conboy, David Schaffer. Oncotarget, Vol 6, No. 14, 11959-11974.
Wen Jiang, Yun Zhang, Lan Xiao, Jamie Van Cleemput, Shao-Ping Ji, Guang Bai, and Xia Zhang, J Clin invest 115(11): 3104-3116.

The above discussion is meant to be illustrative of the principle and various embodiments of the present invention. Although the present invention discloses neurodegenerative combination therapy for Alzheimer's disease the invention can also be used for treating other neurodegenerative diseases such as Parkinson's, ALS, and MS. Numerous variations and modifications will become apparent to those skilled in the art. It is intended that the following claims be interpreted to embrace all such variations and modifications.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method of inhibiting or treating a neurodegenerative disease, or one or more symptoms thereof, comprising: administering to a subject having a neurodegenerative disease an effective amount of a combination of at least two of: i) one or more mitochondria stimulants, ii) one or more antioxidants that cross the blood brain barrier, or iii) one or more neurogenesis agonists,

2. The method of claim 1 where the neurodegenerative disease is Alzheimer's disease.

3. The method of claim 1 wherein the mitochondria stimulant comprises nicotinamide mononucleotide or an analog thereof.

4. The method of claim 1 wherein the mitochondria stimulant is administered in an amount from about 50 milligrams to about 2500 milligrams per day.

5. The method of claim 1 wherein the antioxidant comprises N-acetyl ethyl ester, glutathione ethyl ester, hydroxytyrosol or genestein.

6. The method of claim 1 wherein the antioxidant is administered in an amount from about 25 milligrams to about 7500 milligrams per day.

7. The method of claim 1 wherein the neurogenesis agonist comprises 2-(3-(6-methylpyricin-2-yl)-1H-pyrazol-4-yl)-1,5-naphthyridine, [(6aR)-trans-3-(1,1-dimethylheptyl)-6a, 7,10,10a-tetrahydro-1-hydroxy-6, 6-dimethyl-6H-dibenzo [b,d] pyran-9-methanol] or anatamide.

8. The method of claim 1 wherein the neurogenesis agonist is administered in an amount from about 0.0005 μM to about 20 μM per day.

9. The method of claim 1 wherein the combination is orally administered.

10. The method of claim 1 wherein at least one of the combination is orally administered.

11. The method of claim 10 wherein at least one of the combination is in sustained release dosage form.

12. A method of inhibiting or treating a neurodegenerative disease or one or more symptoms thereof, comprising: administering to a subject having a neurodegenerative disease an effective amount of a combination of at least two of: i) one or more mitochondria stimulants, ii) rapamycin, or iii) one or more antioxidants that cross the blood brain barrier.

13. The method of claim 12 wherein the neurodegenerative disease is Alzheimer's disease.

14. The method of claim 12 wherein the mitochondria stimulant comprises nicotinamide mononucleotide or an analog thereof.

15. The method of claim 14 wherein the mitochondria stimulant is administered in an amount from about 50 to about 2500 milligrams per day.

16. The method of claim 12 wherein the antioxidant comprises N-acetyl ethyl ester, glutathione ethyl ester, hydroxytyrosol or genestein.

17. The method of claim 12 where the antioxidant is administered in an amount from about 25 to about 7500 milligrams per day.

18. The method of claim 17 wherein at least one of the combination is orally administered.

19. The method of claim 15 further comprising administering one or more neurogenesis agonists.

20. A pharmaceutical composition comprising at least two of: i) one or more mitochondria stimulants, ii) one or more antioxidants that cross the blood brain barrier, or iii) one or more neurogenesis agonists or comprising at least two of: a) one or more mitochondria stimulants, b) rapamycin, or c) one or more antioxidants that cross the blood brain barrier.
